(19) United States
(12) Patent Application Publication
(10) Pub. No.: US 2005/0255045 A1
(43) Pub. Date: Nov. 17, 2005

(54) Surgical marking composition and method

(76) Inventor: Eugene A. Woltering, Kenner, LA (US)

Correspondence Address: PATENT DEPARTMENT;TAYLOR, PORTER, BROOKS & PHILLIPS, L.L.P, P.O. BOX 2471, BATON ROUGE, LA, 70821-2471, US

(21) Appl. No.: 10/845,279

(22) Filed: May 13, 2004

(51) Int. Cl. A61K 49/04

(52) U.S. Cl. 424/9.34; 424/9.411

(57) ABSTRACT

A surgical marking system has been developed that can be easily used during surgery to mark an area of the body, e.g., the margins of a tumor resection cavity, for post-operative radiation therapy or subsequent evaluation by CT, MRI, or radiography. This marker system is formed as a semi-liquid solution that is expelled into the resection margins as a stream that quickly polymerizes in situ into a solid or semi-solid strand that adheres to the surrounding tissue. Several of these strands may be placed to outline the cavity surface. One or more of the polymerizing agents contain one or more imageable markers for post-operative imaging or therapy. The method allows a surgeon to outline the margins of a surgical site in all directions. In addition, radioactive isotopes or therapeutic drugs can be added to the marker strands for in situ therapy.

The development of this invention was subject to a contract between the Board of Supervisors of Louisiana State University and Agricultural and Mechanical College, and the United States Department of Veterans Affairs. The Government has certain rights in this invention.

This invention pertains to a new marker system that quickly and easily marks an area inside the body during surgery, e.g., to outline the outer perimeter of a tumor resection cavity, to permit localized therapy and post-operative imaging for subsequent therapy or evaluation.

Following tumor resection, physicians often mark the margins of the tumor resection cavity as an aid for planning post-operative radiation therapy or subsequent evaluation by computed tomography (CT) scans, magnetic resonance imaging (MRI), or radiographs (X-rays). Currently, surgeons typically use multiple metal clips to mark the edges of the resection cavity. To accurately map the irregular borders of a tumor resection cavity with metal clips takes a long time. To decrease the time to mark the borders, surgeons often mark only the 12, 3, 6, and 9 o'clock positions on the anterior surface of the resection. This method does not adequately indicate the depth of the resection margins, nor the lateral, medial, caudal, and cephalad portions of an irregular wound. Additional disadvantages exist with metal clips: First, metal clips can cause scatter artifact in CT scans. Second, this marking process is time-consuming, and thus costly to the patient. Third, the metal clips are not contiguous markers, and only mark representative areas of the cavity. Fourth, metal clips are permanent, not bio-absorbable. Fifth, some metal markers may prevent subsequent MRI scans, at least in the immediate post-operative period (within about three months) when radiation planning is most needed. Sixth, the metal clips can come loose and migrate to other areas of the body.

Surgeons would also have reasons to mark other areas of the body at the time of surgery for later evaluation by remote imaging. For example, surgeons may want to mark the site of a vascular anastomosis, to help locate the site in the event of a pseudoaneurysm formation. Also, for future evaluation, it would be helpful to mark a cavity from the removal of a cyst or abscess (e.g., a hydatid cyst caused by Echinococcosis or a cyst caused by Entamoeba), a bone fracture repair site, a nerve anastomosis site, a gut anastomosis site, a bronchial anastomosis site, or the site of an implanted tissue transplant. In particular, marking the site of a gut anastomosis would be useful, especially if the repair was hand-sewn rather than stapled. Marking the site allows later imaging to determine how the actual anastomosis moves in the abdomen.

There exists a need for a bio-absorbable, inexpensive, fast system for marking areas of the body during surgery to permit postoperative imaging, e.g., marking the margins of a tumor resection cavity for postoperative evaluation or therapy.

A number of procedures and devices for marking and locating either skin or tissue locations are known. Some examples are the following:

U.S. Pat. No. 5,147,307 discloses a device and method for marking the surface of the skin prior to a diagnostic or therapeutic procedure by using thin-edged, tapered patterning elements that are impressed into the skin.

U.S. Pat. No. 5,192,270 discloses a device for marking the skin for injection sites using a pigment or dye deposited into a dimple on the protective cover of the needle.

U.S. Pat. No. 5,221,269 discloses a device for localizing a nonpalpable breast lesion using a helical wire coil that locks into position around the lesion. The coil is inserted using a hypodermic needle.

U.S. Pat. No. 5,902,310 discloses a device to deliver through the skin a permanent marker to selected tissue locations, for example, a radiographic clip in the form of a surgical staple.

U.S. Pat. No. 6,371,904 discloses a subcutaneous cavity-marking device for either a bioabsorbable or non-bioabsorbable marker that self-expands after delivery to fill the cavity left by a lesion resection or a biopsy.

U.S. Pat. Nos. 6,427,081 and 6,161,034 disclose detectable markers that are prepared in advance and then are later introduced to fill a cavity created by removal of a biopsy specimen to mark the location of the biopsy site. The markers remain at the site for about 2 weeks but are gone by 5 to 7 months. The markers can be combined with other compounds to decrease absorption or to increase tissue adhesion adjacent to the biopsy cavity. It was noted that the introduced markers might distend or stretch the biopsy cavity somewhat.

I have discovered a surgical marking system that can be easily used during surgery to mark an area of the body, e.g., the margins of a tumor resection cavity, for post-operative therapy or subsequent evaluation by CT, MRI, or radiography. This marker system is not prepared in advance, but instead is formed as a semi-liquid solution that is expelled into the resection margins as a stream that quickly polymerizes in situ into a solid or semi-solid strand that adheres to the surrounding tissue. Several of these strands may be placed to outline the cavity surface. Embodiments of this marker system may be based on known agents that quickly polymerize into a solid when mixed, e.g., albumin mixed with glutaraldehyde, fibrinogen mixed with thrombin, or any of several synthetic polymers mixed with water. One or both of the polymerizing agents contain one or more imageable markers for post operative imaging or therapy. A short polymerization time makes the marker system a fast and easy method for a surgeon to outline the margins of the cavity immediately after removal of the tumor. In one application method, two solutions in separate syringes are mixed just prior to being expelled as a single stream to the resection cavity. For best results, the resulting mixed solution should polymerize and harden in about 30 sec to about 1 min. The concentration of the marker can be adjusted to achieve a final optical density that will contrast with the tissue's intrinsic optical (radiological) density. The polymerizing system can be chosen based on the density of the solid formed and the desired residence time in the body. The method allows a surgeon to outline a surgical site in all directions. In addition, radioactive isotopes or therapeutic drugs could be added to the marker strands for in situ therapy.

One such two-solution/polymerizing system is a protein-based hydrogel, based on the interaction of serum albumin and glutaraldehyde. The system produced a solid product with a consistency comparable to bone and a residence time of months to years. One similar commercial product, without a marker agent, is marketed under the trademark BioGlue® (CryoLife®, Inc., Kennesaw, Ga.). It is known to have strong adhesive and cohesive properties, to be biocompatible, and to polymerize within 20 to 30 sec. The current use of BioGlue® is primarily to help repair or reinforce vessel anastomoses. In embodiments of the present invention, the polymerized product was made more pliable by the addition of a plasticizer, glycerol. Other plasticizers that may be used are based on various vegetable oils or on esters of fatty acids derived from vegetable oils, including soybean oil, linseed oil, coconut oil, canola oil, palm oil, castor oil, stearic acid, oleic acid, palmitic acid, sebacic acid, etc.

A second embodiment is based on the interaction between fibrinogen and thrombin to form a fibrin-thrombin solid matrix, analogous to a blood clot. There are several commercial products for stopping blood flow based on similar systems, e.g., Tisseel VH Fibrin Sealant (Baxter Healthcare Corporation, Fremont, Calif.). The polymerized product is softer and more pliable, and has a shorter residence time (from about six to eight weeks) than that of the albumin/glutaraldehyde system. This product is useful, once mixed with a marker, to outline the perimeter of tissue lesions in soft, pliable tissues such as breast tissue, where the post-operative imaging is conducted very soon after tumor resection. The polymerization time is about 30 sec.

A third embodiment is based on biocompatible, biodegradable, branched thermoplastic polymers, as described in U.S. Pat. No. 6,528,080. This patent discloses the use of these polymers for drug implants or other controlled release devices in the body. One commercial product is ATRIGEL® (Atrix Laboratories, Inc., Fort Collins, Colo.). Polymerization occurs when the monomer is mixed with water. The polymerization time is usually from 60-120 seconds, but can be adjusted by altering concentrations of the components. The resulting product is somewhat pliable and soft, with a residence time less than one year.

Desirable properties of the two-solution/polymerizing system used in this invention include the following: The solutions should be made sterile and pyrogen free. The resulting product should be bioabsorbable and non-toxic. The polymerization time should be relatively short, preferably from about 10 sec to about 180 sec, more preferably from about 30 sec to about 120 sec, and most preferably from about 30 to about 60 sec. The system should still polymerize when a marker is added in concentrations useful for imaging.

One or more markers are added to the polymerizing systems to enhance imaging, including for example, X-ray, CT scan, mammography, fluoroscopy, other roentgenologic means, magnetic resonance imaging (MRI), ultrasound, Doppler, optical imaging of flourescein, or other known imaging techniques. The marker may, for example, comprise a contrast agent based on a radioactive substance, or high Z (atomic number) atoms, such as a high Z metal salt (e.g., silver or iron salt), iodine, gadolinium, stainless steel, powdered metals such as titanium, iron, etc. In one embodiment, the marker is a material with a different magnetic density, e.g., iron powder, from that of the tissue (e.g., superparamagnetic iron oxides (SPIOs) and ultrasmall superparamagnetic iron oxides (USPIOs)). If placed properly, the marker could even be a fluorescent compound or could be flourescein that could be imaged optically under a blue ultraviolet light. The marker should be imageable by the postoperative imaging system desired. For example, three remote imaging systems commonly used for radiation therapy are X-rays, CT scans, and MRI scans. For imaging in all three, the markers used could include iodine and gadolinium. The marker, at the concentration used, should be nontoxic, sterile, pyrogen-free, and relatively inert biologically. The marker could be a liquid or a suspension of particles, so long as it does not inhibit the polymerization of the product. The marker could also act as a plasticizer, e.g., ETHIODOL® (Savage Laboratories, Melville, N.Y.), the ethyl ester of an iodinated fatty acid of poppyseed oil. The combination of the marker with the mixture of the polymerizing solutions should be a product that is easy to apply as a steady stream, but having a consistency that allows it to be applied much like a thread, string or strand of semi-liquid material. This stream polymerizes after contacting the tissue to form a solid strand that adheres to the tissue.

The choice of polymerization system will depend on the desired residence time in the tissue, and on the desired density of the resulting product. For example, if the tumor was removed from breast tissue, the fibrin/thrombin system might be used because it forms a pliable product that has a short residence time. The desired residence time depends on the length of time that a discernable image is required. If radiation therapy is begun soon after the tumor removal, then the desired residence time is short, as in breast tumor removal. However, when cancers are removed from the abdominal cavity, radiation or other therapy is often delayed until the incision heals. In that case, a longer residence system, either the ATRIGEL® or albumin/glutaraldehyde system, would be better.

One method of application of any of the above systems is to use two syringes that feed into a single mixing chamber prior to delivery, for example, to mark the margins of a tumor resection cavity. This marker system should be mixed and applied immediately after tumor resection and after adequate hemostasis. The resection cavity may be dried to some extent before application. One syringe is loaded with one or more markers plus one of the two polymerizing solutions, while the second syringe is loaded with the other polymerizing solution, and optionally, with one or more markers as well. The solutions then flow into a mixing chamber, and the resulting mixed solution is applied in a stream of material that polymerizes and adheres to the resection cavity walls. The resection cavity is lined with strings or strands of polymer that are also placed around the rim of the cavity. Preferably a meshwork is created by laying strands across the cavity in different directions. One way to visualize this system is to envision lining a hollowed-out grapefruit half with a “string” that adheres to the grapefruit. The resulting meshwork of strands outlines the shape of the cavity, including its perimeter and depth, but does not fill the cavity. Because the solutions polymerize and adhere quickly, any strand of the meshwork that does not adhere to tissue can be corrected, either by adding new strands or by tacking the strand in place. Subsequent postoperative evaluation with an imaging method sensitive to the marker is used to image the outline of the resection cavity. An advantage of this system is that the margins of the resection cavity are preserved by the meshwork of strands regardless of any change in shape of the cavity after the operation is over and the patient resumes normal activity. This system of outlining the margins of the cavity soon after resection preserves the margins of the cavity better than filling the cavity with material, since filling the cavity may enlarge or distort the margins of the cavity, especially when the patient resumes activity. It is the actual margins of the tumor resection that are important for directing post-operative radiation therapy.

This new marker system is also useful to mark other areas during surgery, for instance, areas of anastomosis. For example, a surgeon could mark the area of a vascular anastomosis to monitor for pseudoaneurysms. In addition, this marker system could be used for postoperative imaging of the sites of cyst or abscess removal, lymph node resection, bone fracture repair, nerve anastomosis, bronchial anastomosis, or gut anastomosis. Marking the site of a gut anastomosis with a single strand or a series of strands would reinforce the anastomosis, and allow a determination of the actual movement of the anastomosis within the abdomen. This marking system could easily be applied using a coding system to indicate areas of a high risk of tumor recurrence or location along the gut of multiple anastomosis sites (e.g., one stripe for most proximal, two stripes for a more distal, and three stripes for the most distal anastomosis). The speed and versatility of this marking system gives a surgeon great flexibility in coding or marking a surgical area that could be followed post-operation with imaging techniques.

In an alternative embodiment, the polymerized strand may also contain therapeutic compounds. The marker system could be used for any surgical site for which postsurgical evaluation is beneficial and for which therapeutic compounds can be applied in situ. For example, the strand may contain radioisotopes, for example, radiolabeled iodine (e.g., a radiolabeled ETHIODOL®), to provide in situ radiation treatment by the actual meshwork created by the strands lining the cavity. The density of the meshwork would be chosen based on the radius of activity of the radioisotope. In another therapeutic embodiment, a polymerized strand may contain a marker and a boron cage such that when the strand is exposed to a neutron beam, the boron captures the neutrons and then radioactively decomposes. Moreover, rows of strands could be equally spaced by using a device with multiple mixing and application units so that multiple strands could be applied at one time at a set distance from each other.

The polymerized strand could also contain drugs (i.e., biologically active agents) for in situ controlled drug therapy, especially in the marker systems based on ATRIGEL® or fibrin/thrombin or other similar polymerizing systems. See, U.S. Pat. No. 6,528,080. The “biologically active agents” are compounds that could be used for treatment, prevention, diagnosis, cure or mitigation of a disease or illness, a substance that affects local structure or function of the body (a “biologic response modifier”), or pro-drugs, which become active or more active after placement in the body. These agents include, by way of example, anti-infective, antibacterial, antimicrobial, antifungals, antiviral, anti-parasitic, anti-inflammatory, anti-angiogenesis, analgesic and cytotoxic agents, or beneficial hormones (e.g., growth hormone) or other peptides or proteins. For a more complete listing, see U.S. Pat. No. 6,528,080. For example, a marker system containing antibiotics could be used to mark and treat the cavity after removal of abscess. A marker system containing anti-parasitic compounds could be used to mark and treat a cavity after removal of a cyst caused by a parasite, e.g., Echinococcosis granulosa or Entamoeba histolytica. Moreover, the marker system could contain certain biologic response modifiers, e.g., a growth hormone in a marker for a bone fracture site to speed recovery.

### EXAMPLE 1

An Albumin/Glutaraldehyde Polymerizing System with Marker

In one embodiment, an iodinated marker was added to a polymerizing system based on glutaraldehyde and albumin. Equal volumes of albumin (40% w/v in water; human albumin Fraction V, Sigma A-1653; Sigma Chemical Co., St. Louis, Mo.; other sources of albumin could be used, e.g., bovine) and glutaraldehyde (10% v/v in water; Sigma-G-6403) were used to make the polymerized product. Each solution was loaded into one barrel of a dual-barrel syringe (EndoRez®; Ultradent Products, South Jordan, Utah), or into two syringes connected with a three-way stopcock (Baxter, Deerfield, Ill.). When the solutions were mixed in equal volumes and expressed in a thin stream, polymerization occurred. For imaging, glutaraldehyde was combined with ISOVUE® 300 (iopamidol; a tri-iodinated, non-ionic, water-soluble marker; ISOVUE® 300 contains 300 mg iodine/ml; Bracco Diagnostics, Princeton, N.J.). The resulting product was easily seen in an X-ray film. Previous experiments had shown that this marker should be added to the glutaraldehyde rather than to the albumin solution for polymerization to occur properly.

Experiments were conducted to determine the effect on polymerization time of changing concentrations of glutaraldehyde (from 0.5 to 5%) and of ISOVUE® 300 (from 0.5 to 15%). Solutions were prepared as above, and the polymerization times measured. Albumin was 20% in all cases. The results are given in Table 1.

TABLE 1
Polymerization Time for Different Final Concentrations
of Glutaraldehyde and Marker

| | 0% | 0.5% | 15% |
|---|---|---|---|
| Glutaraldehyde | ISOVUE ® 300 | ISOVUE ® 300 | ISOVUE ® 300 |
| Concentration | Time (sec) | Time (sec) | Time (sec) |
| 5% | 8 | 7 | 12 |
| 4% | 15 | 12 | 20 |
| 2.5% | 23 | 20 | 30 |
| 2% | 30 | 25 | 35 |
| 1% | 60 | 75 | 90 |
| 0.5% | 300 | 240 | 915 |

As shown in Table 1, decreasing the glutaraldehyde concentration had a dramatic effect on the polymerization time, e.g., about 10 sec with 5%, and over 4 min with 0.5% glutaraldehyde. Increasing the marker concentration only somewhat increased the polymerization time.

To make the resulting product more pliable, glycerol was added to the glutaraldehyde solution. In this experiment, equal parts of 40% albumin were mixed with 5% glutaraldehyde with 5% glycerol (v/v with water), and 50% ISOVUE® 300. The resulting product contained 20% albumin, 2.5% glutaraldehyde, 2.5% glycerol, and 25% ISOVUE® 300. The product was more pliable, and the polymerization time was about 60 sec.

From the above experiments, the preferred concentration of glutaraldehyde is greater than about 1% to achieve a polymerization time less than or equal to 90 sec. This marker system could be used to mark the perimeter of a lesion that has been removed such that the perimeter could be detected by various radiographic means. In addition, as shown below in Example 5, the use of other markers allows detection by other imaging methods, including CT or MRI. Gadolinium or other markers, such as the non-ionic contrast agents widely used for CT or MRI scanning, could also be used. Non-water soluble contrast agents could also be used to create a suspension rather than a solution of these agents (e.g., powdered stainless steel or titanium).

### EXAMPLE 2

Biodegradable, Thermoplastic Polymer Polymerizing System and Marker

In a second embodiment of the invention, experiments were conducted on adding a marker to a commercial, biodegradable, branched thermoplastic polymer, ATRIGEL®, from Atrix Laboratories, Inc. (Fort Collins, Colo.). ATRIGEL® is a bioabsorbable, flowable polymeric formulation composed of 36.7% poly(DL-lactide)(PLA) dissolved in 63.3% N-methyl-2-pyrrolidone (NMP). The product solidifies when it comes in contact with water. The contrast agent (ISOVUE® 300) was lyophilized to dryness, and then 0.5 g dissolved in a solvent solution of NMP and water. The ATRIGEL® was mixed with the contrast-solvent solution using a double-barrel syringe or three-way stopcock as described in Example 1. The mixture was then expressed as a thin stream, and the polymerization time measured. Experiments were conducted to optimize concentration of the organic solvent and of water.

Table 2 gives the polymerization time for various percentages of final water content from 5 to 50%. As shown in Table 2, lower water content resulted in longer times to solidify.

TABLE 2
Polymerization Time for Various Solutions

| | % FINAL WATER CONTENT | | | | |
|---|---|---|---|---|---|
| | 50% | 40% | 20% | 10% | 5% |
| ISOVUE ® 300 (g) | 0.5 | 0.5 | 0.5 | 0.5 | 0.5 |
| Water (ml) | 1 | 0.8 | 0.4 | 0.2 | 0.1 |
| NMP (ml) | 0 | 0.2 | 0.6 | 0.8 | 0.9 |
| % ISOVUE ® 300 that is Water | 100 | 80 | 40 | 20 | 10 |
| Total Volume ISOVUE ® 300 (ml) | 1.0 | 1.0 | 1.0 | 1.0 | 1.0 |
| Volume ISOVUE ® 300 Used (ml) | 0.5 | 0.5 | 0.5 | 0.5 | 0.5 |
| Volume of ATRIGEL ® Used (ml) | 0.5 | 0.5 | 0.5 | 0.5 | 0.5 |
| % ISOVUE ® 300 (w/v) | 25 | 25 | 25 | 25 | 25 |
| Time to Solidification | <5 sec | <5 sec | 12 sec (not | >3 min (not | Never |
| | | | homogenous) | homogenous) | |

The optimal solution of this system contains about 20% water, with enough contrast to exceed the optical density of the surrounding tissue (e.g., 25% ISOVUE ® 300 or more).

### EXAMPLE 3

Fibrin/Thrombin Polymerizing System and Marker

In a third embodiment, a marker was combined with a system using thrombin and fibrinogen. In this experiment, an equal volume of fibrinogen (from 5 to 10 mg/ml; Fibrinogen, Type I-S from bovine plasma, Sigma F8630) was dissolved with a marker solution (30% or 60% ISOVUE® 300) for the first solution. The second solution contained thrombin (25 to 100 Units; Thrombin from bovine plasma, Sigma T-7513). These two solutions were mixed using either a double-barrel syringe or a three-way stopcock as described above in Example 1. The mixture was expressed as a thin stream. Polymerization occurred in about 30 sec to about 60 sec. This polymerized meshwork should remain intact in the body for several days to weeks.

### EXAMPLE 4

Imaging of Model Tumor Resection Cavity

Each of the marker systems described in Examples 1, 2, and 3 were tested and scanned by plain radiography, CT scans, and MRI scans at the Medical Center of Louisiana at New Orleans. All scans were conducted using standard clinical scanning parameters. To model a tumor resection cavity, a gelatin phantom was created. Gelatin was chosen because it approximates the density of body tissue. The gelatin was dissolved according to the manufacturer's instructions. One 6 oz package of JELL-O® (Kraft Foods, Inc.; Northfield, Ill.) was mixed with 800 ml boiling water. An initial layer of gelatin was created by adding 400 ml gelatin solution to a plastic 9 L container and allowed to gel. A marble was then placed on top of this layer, and another 150 ml gelatin solution added to partially cover the marble. Once the solution gelled, the marble was removed, and the resulting model cavity was lined with polymerizing strands using one of the above polymerizing/marker systems. After the strands polymerized, an additional layer of gelatin (250 ml gelatin solution) was added to cover the model cavity with the newly polymerized strands. The gelatin model was then imaged by X-ray, CT and MRI. The images were downloaded onto CD-RW discs for analysis. Each marker system produced an accurate image of the margins of the initial cavity. (Data not shown).

For the MRI scans, the marker in each polymerizing system was changed to a gadolinium complex. In particular, gadopentetate dimeglumine was used (MAGNEVIST®; 0.5 mol/L solution of 1-deoxy-1-(methylamino)-D-glucitol dihydrogen [N,N-bis[2-[bis(carboxymethyl)amino]ethyl]-glycinato-(5′)-]gadolinate; BERTEX® Laboratories; Wayne, N.J.).

### EXAMPLE 5

A Polymerizing System with Two Markers

To test whether two different markers could be simultaneously used in the above polymerizing systems, both ISOVUE® 300 and MAGNEVIST® were added to each of the three different polymerizing systems discussed in Examples 1, 2, and 3. Before adding the markers to the ATRIGEL® polymerizing system, a set volume of ISOVUE® 300 (0.6 ml) and MAGNEVIST® (0.3 ml) was lyophilized to dryness, then reconstituted with the appropriate solvent system. The polymerizing systems were mixed as before using solutions as described below, and the resulting polymerizing strands used to outline a model tumor resection cavity as described in Example 4.

For the albumin/glutaraldehyde system, syringe one contained 40% albumin (1 ml), and syringe two contained 5% glutaraldehyde (0.1 ml), ISOVUE® 300 (0.6 ml), and MAGNEVIST® (0.3 ml). Equal volumes (1 ml from each syringe) were mixed to form 2 ml mixed solution, which was used to produce a stream to outline the model tumor resection cavity. The final product was thus 20% albumin, 2.5% glutaraldehyde, 30% ISOVUE® 300, and 15% MAGNEVIST®.

For the ATRIGEL® system, syringe one contained 37% 85:15 ATRIGEL® (1 ml), and syringe two contained 0.8 ml NMP, 0.2 ml water, ISOVUE® 300 (0.6 ml dried and reconstituted with an aliquot from the 0.8 ml NMP), and MAGNEVIST® (0.3 ml dried and reconstituted with an aliquot from the 0.8 ml NMP). Equal volumes (1 ml from each syringe) were mixed to form 2 ml mixed solution, which was used to produce a stream to outline the model tumor resection cavity. The final product was thus 18.5% ATRIGEL®, 10% water, 30% ISOVUE® 300, and 15% MAGNEVIST®.

For the fibrinogen/thrombin system, syringe one contained thrombin (100 U/ml; 1 ml), and syringe two contained fibrinogen (100 mg/0.1 ml water), ISOVUE® 300 (0.6 ml), and MAGNEVIST® (0.3 ml). Equal volumes (1 ml from each syringe) were mixed to form 2 ml mixed solution, which was used to produce a stream to outline the model tumor resection cavity. The final product was thus 50 Units thrombin, 50 mg fibrinogen, 30% ISOVUE® 300, and 15% MAGNEVIST®.

The polymerization times for all three marking system with two markers were not noticeably different from those seen when only one marker was added. All systems polymerized between about 30 sec and about 60 sec.

Each model cavity was then scanned using plain radiographs (X-ray), CT scans, and MRI scans. The systems were scanned at 48 hr after the initial lining of the model cavity, and then again at 10 days after the initial lining. At 48 hr all samples were clearly visible and the strands distinct. At 10 days, the strands in the samples lined with ATRIGEL® or glutaraldehyde/albumin were still clearly visible. However, the strands in the samples lined with fibrin/thrombin, although still visible, were more diffuse than the other two systems. It is believed that the fibrin/thrombin system in tissue would not diffuse as rapidly as in this gelatin model.

The complete disclosures of all references cited in this specification are hereby incorporated by reference. In the event of an otherwise irreconcilable conflict, however, the present specification shall control.

1. A kit comprising a first composition, a second composition, a marker, and a mixer; wherein:
(a) neither said first composition alone, nor said second composition alone, readily polymerizes under ambient conditions;
(b) said mixer comprises compartments that are adapted to hold a first liquid and a second liquid, and to keep the first and second liquids separate from each other; and then, at a time selected by the user, to mix at least some of the first liquid with at least some of the second liquid, and to extrude the resulting mixture;
(c) the first liquid comprises said first composition, or the first liquid comprises said first composition mixed with a first solvent; and the second liquid comprises said second composition, or the second liquid comprises said second composition mixed with a second solvent; wherein the first and second solvents may be the same or different;
(d) the first liquid comprises said marker; or the second liquid comprises said marker; or said mixer comprises compartments to hold the marker separate from the first and second liquids, and then to mix said marker with the first and second liquids; whereby, the mixture extruded by said mixer comprises said marker;
(e) the extruded mixture is initially liquid, but polymerizes into a hardened solid or semi-solid over a period between about 10 seconds and about 180 seconds after extrusion;
(f) the extruded mixture, when liquid, will adhere to living tissue; and after hardening, will stay adhered to the tissue for a period between about two weeks and about five years; but the extruded mixture is ultimately biodegradable after the passage of sufficient time;
(g) the first composition, the second composition, and the marker are all sterilized, and are all substantially free from pathogens;
(h) the extruded mixture, when hardened, is nonpyrogenic and nontoxic; and
(i) said marker substantially enhances imaging of the hardened, extruded mixture as compared to the imaging that is otherwise obtainable for the tissue to which the mixture adheres; by at least one imaging technique.

2. A kit as recited in claim 1, wherein said imaging technique is selected from the group consisting of radiography, autoradiography, computed tomography, fluoroscopy, ultrasound, Doppler, magnetic resonance imaging, and an optical detector of activated flourescein.

3. A kit as recited in claim 1, wherein said first composition comprises albumin and said second composition comprises glutaraldehyde.

4. A kit as recited in claim 1, wherein said first composition additional comprises a plasticizer, or said second composition additionally comprises a plasticizer.

5. A kit as recited in claim 4, wherein said plasticizer is glycerol.

6. A kit as recited in claim 1, wherein said first composition comprises thrombin and said second composition comprises fibrinogen.

7. A kit as recited in claim 1, wherein said first composition comprises a branched thermoplastic polymer and said second composition comprises a polymerizing solvent.

8. A kit as recited in claim 1, wherein the extruded mixture polymerizes into a hardened solid over a period between about 30 seconds and about 120 seconds after extrusion.

9. A kit as recited in claim 1, wherein the extruded mixture polymerizes into a hardened solid over a period between about 30 seconds and about 60 seconds after extrusion.

10. A kit as recited in claim 1, wherein said marker is selected from the group consisting of a radioactive isotope, iodine, titanium, gadolinium, stainless steel, a fluorescent compound, flourescein, a salt of another high-atomic number metal, or a powder of another high- atomic number metal.

11. A kit as recited in claim 1, wherein said marker comprises iodine.

12. A kit as recited in claim 1, wherein said marker comprises gadolinium.

13. A kit as recited in claim 1, wherein said marker comprises gadolinium and iodine.

14. A kit as recited in claim 1, wherein said mixer is adapted to extrude said mixture in multiple streams simultaneously.

15. A kit as recited in claim 1, wherein said first composition or said second composition additionally comprises boron cages.

16. A kit as recited in claim 1, wherein said first composition or said second composition additionally comprises one or more biologically active agents.

17. A kit as recited in claim 16, wherein said biologically active agent is selected from the group consisting of anti-infective, antibacterial, antimicrobial, antifungal, antiviral, anti-parasitic, anti-inflammatory, anti-angiogenic, analgesic and cytotoxic agents, beneficial hormones or other peptides, and other biologic response modifiers.

18. A method for marking the location of a surgically-exposed area in the interior of living tissue, said method comprising applying an extruded liquid mixture produced by the kit of claim 1 to at least a portion of the area, and allowing the extruded mixture to adhere to and harden upon the tissue; whereby the marker within the hardened mixture will allow the location to be imaged even after the once surgically-exposed area has been closed.

19. A method as recited in claim 18, wherein the margins of a resection cavity in tissue are marked with a network of the extruded mixture.

20. A method as recited in claim 19, wherein the resection cavity is a tumor resection cavity.

21. A method as recited in claim 19, wherein the resection cavity is not filled with the extruded mixture, but the instead is marked only on the periphery of the cavity.

22. A method as recited in claim 18, wherein the extruded mixture comprises one or more radioisotopes suitable for radiation therapy; whereby, in addition to providing enhanced imaging properties, the extruded mixture also supplies radiation therapy to tissue in the vicinity of the extruded mixture.

23. A method as recited in claim 18, wherein the extruded mixture comprises one or more biologically active agents; whereby, in addition to providing enhanced imaging properties, the extruded mixture also supplies an agent for modifying tissue in the vicinity of the extruded mixture.

24. A method as recited in claim 23, wherein said biologically active agent is selected from the group consisting of anti-infective, antibacterial, antimicrobial, antifungal, antiviral, anti-parasitic, anti-inflammatory, anti-angiogenic, analgesic and cytotoxic agents, beneficial hormones or other peptides, and other biological response modifiers.

25. A method as recited in claim 18, wherein the mixture is extruded in multiple streams from said mixer.

26. A method as recited in claim 18, wherein the marked area comprises the site of a removed cyst, the site of a removed abscess, the site of a vascular anastomosis, the site of a fracture repair, the site of a nerve anastomosis, the site of a gut anastomosis, the site of a bronchial anastomosis, or the site of an implanted tissue transplant.
